(19) United States
(12) Patent Application Publication
(10) Pub. No.: US 2025/0241840 A1
(43) Pub. Date: Jul. 31, 2025

(54) ORALLY ADMINISTRATED COSMETIC COMPOSITIONS TO TREAT SKIN DRYNESS

(71) Applicant: IIAA Limited, London (GB)

(72) Inventors: Lorraine PERRETTA, London (GB); Paul NAUDE, London (GB); Tanya MEYER, London (GB)

(21) Appl. No.: 19/181,821

(22) Filed: Apr. 17, 2025

Related U.S. Application Data
(63) Continuation of application No. PCT/GB2023/052710, filed on Oct. 19, 2023.

(30) Foreign Application Priority Data
Oct. 21, 2022 (GB) 2215592.3

(51) Int. Cl. A61K 8/73; A61K 8/19; A61K 8/36; A61K 8/42; A61K 8/67; A61Q 19/00

(52) U.S. Cl. CPC A61K 8/735; A61K 8/19; A61K 8/361; A61K 8/42; A61K 8/671; A61Q 19/007; A61K 2800/92

(57) ABSTRACT

Provided is a kit, comprising: a first composition for oral administration, comprising hyaluronic acid, one or more ceramides and manganese; and a second composition for oral administration, comprising retinol or a derivative thereof, one or more omega-3 fatty acids and one or more omega-6 fatty acids. Also provided are related products, methods and uses.

### RELATED APPLICATIONS

This application is a U.S. continuation application claiming priority to International patent application PCT/GB2023/052710, filed Oct. 19, 2023, which claims priority to UK Patent Application Ser. No. GB2215592.3, filed on Oct. 21, 2022. The contents of the above-identified applications are hereby incorporated by reference in their entireties.

### FIELD OF THE INVENTION

The present invention concerns oral compositions. More particularly, but not exclusively, this invention concerns a kit, comprising: a first composition for oral administration, comprising hyaluronic acid, one or more ceramides and manganese; and a second composition for oral administration, comprising retinol or a derivative thereof, one or more omega-3 fatty acids and one or more omega-6 fatty acids. The invention also concerns related products and methods.

### BACKGROUND OF THE INVENTION

Various factors contribute to skin wrinkles and to dry skin. These include harsh soaps; repeated exposure to chlorinated water (such as is found in swimming pools); chronic sun exposure; seasonal changes (cold and dry weather); ageing; allergies; and nutritional insufficiencies. There are also dermatologic diseases characterized by dry skin and/or itchy skin, such, for example, as dermatitis.

Consequently, there is a need for means to alleviate the depth and width of skin wrinkles and to prevent or treat dry skin. Conventionally, compositions comprising one or more humectants and/or one or more emollients have been applied topically to prevent or treat dry skin and to reduce the depth and width of skin wrinkles. For example, moisturizers, balms, oils and butters have been applied topically to the skin and different types of composition may be administered to different areas of the skin of the human body.

More recently, attention has shifted in the cosmetic field to biochemical effects of specific individual active ingredients on skin cells and on their extracellular matrix. In this approach, a formulation may comprise one or two active ingredients together with inactive excipients and/or with conventional emollients and humectants. Yet this approach may lead to a disruption in the homeostasis of the skin, particularly if compositions containing just a single active ingredient are repetitively applied topically. Some end users of topical compositions find it difficult to apply a suitable concentration of product topically to their skin, and may overestimate the quantity required for the desired effect, leading to side effects. Moreover, different skin types react differently to different ingredients, due to different phenotypes. Thus, while one user may be able to use a composition having one or two active ingredients to provide a desired cosmetic effect without significant side effects, another user may find that they are unable to do so because the composition does not deliver the correct active ingredients their individual skin requires. A composition containing only one or two active ingredients may disrupt the homeostasis of the skin layers, such as, for example, that of the stratum corneum and the extracellular matrix of the epidermis. For instance, one or both of the production of sebum and the synthesis of collagen may be disrupted. Additionally or alternatively, the skin microbiome may be disturbed.

A further problem with topical compositions, which sit on the surface of the stratum corneum, is their tendency to wear away quickly, for example if skin to which a composition has been applied is brushed against, or if it becomes wet.

The present invention seeks to solve or mitigate the above-mentioned problems.

### SUMMARY OF THE INVENTION

The present inventors have found that by providing a composition for oral administration having a large number of active ingredients which can work in synergy with each other to preserve or restore hydration in the stratum corneum and the extracellular matrix of the epidermis, it is possible to prevent, treat or mitigate skin dryness. This can be achieved without disrupting the homeostasis of the skin layers, because it does not involve just one or just two active ingredients, but a blend of active ingredients which complement each other and balance each other out.

Moreover, this may solve or mitigate the problem that individual users' genetic, phenotypic, and environmental factors differ, because a blend of several active ingredients on average provides the right ingredient, or balance of ingredients, for a range of different skin types.

Suitably, products and methods in accordance with the present disclosure may provide an improvement in the composition of skin sebum. The term sebum, as used herein, may refer to an oily secretion of the sebaceous glands, which forms an occlusive coating on the surface of the stratum corneum, thus protecting the skin from the evaporation of water. Improvement in the composition of the sebum is thought to enhance skin hydration, leading to an improved appearance in addition to improved health of the layers of the skin.

Furthermore, administration of compositions orally means more precise dosages can be prescribed, such as by providing directions to take a certain number of capsule dosage forms at defined time intervals. In contrast, dosages provided by a topical composition may be dependent on the skin area covered, the thickness of the skin, and the contact time (amongst other factors). Studies have shown that a topical product may be administered in widely varying dosages from one consumer to another as a result of guesswork and/or human error.

Thus, products (such as kits and compositions) and methods according to one or more (such as all) aspects of the present disclosure may be for reducing skin dryness. Put another way, they may be for increasing skin hydration. It will be appreciated that the terms “reducing skin dryness” and “increasing skin hydration” may be used interchangeably herein, and may be used to refer to the water content and/or the ability to retain water of one or more layers of the skin, particularly human skin, such as the epidermis. A reduction in skin dryness, corresponding to an increase in skin hydration, may be quantified. For example, skin hydration may be quantified. For example, by “increasing skin hydration” herein may be meant an increase in hydration (such as when measured using a Corneometer obtainable from Courage & Khazaka Electronic GmbH of Mathias-Brüggen-Straße 91, 50829 Köln, Germany) of at least 10%; for example, by from 10 to 60%. Additionally or alternatively, a decrease in transepidermal water loss (TEWL) may be used as an indicator of an increase in skin hydration/a reduction in skin dryness. For example, TEWL (such as when measured using a Tewameter obtainable from Courage & Khazaka Electronic GmbH) may be reduced by at least 5%; by at least 10%; or by at least 15%.

As disclosed herein, it has been found to be possible to prevent, treat or mitigate skin dryness in a surprisingly short amount of time, for example within three months, or even within one month.

The term wrinkle is well known in the art and refers to a crease in the skin surface. As described in Example 5 hereinbelow, the inventors have found that products (such as kits and compositions) and methods according to one or more (such as all) aspects of the present disclosure can reduce the width and depth of skin wrinkles. Surprisingly, the inventors observed a synergistic effect, as between the components of the disclosed composition, on wrinkle depth and width of the skin of the cheek. Without wishing to be bound by theory, the cheek is thought to be the largest single area of skin exposed to the environment and weather year-round, so that it may be particularly difficult to improve hydration, and to reduce wrinkle depth and width, of the skin of the cheek. Thus, the findings of Example 5 suggest that the disclosed composition has a particularly advantageous effect on wrinkles.

The findings of Example 5 suggest that the disclosed composition is able to produce this effect on wrinkles without causing irritation of the skin. To the contrary, it improves skin hydration and decreases erythema (redness).

Therefore, products (such as kits and compositions) and methods according to one or more (such as all) aspects of the present disclosure are preferably for reducing the depth and width of skin wrinkles. Yet more preferably, they are for reducing the depth and width of skin wrinkles while also increasing skin hydration and reducing skin erythema.

The reduction of wrinkle depth and width may be quantified. By “reducing wrinkle depth” herein may be meant decreasing wrinkle depth (for example, as measured using a Visioscan obtainable from Courage & Khazaka Electronic GmbH) by at least 10%, for example by between 10 and 30%. Similarly, by “reducing wrinkle width” herein may be meant decreasing wrinkle width (for example, as measured using a Visioscan obtainable from Courage & Khazaka Electronic GmbH) by at least 10%, for example by between 10 and 30%.

As disclosed herein, it has been found to be possible to reduce the depth and width of skin wrinkles in a surprisingly short amount of time, for example within three months, or even within one month.

Additionally or alternatively, skin roughness (such as when measured using a Visioscan obtainable from Courage & Khazaka Electronic GmbH) may be decreased by at least 5%, for example by from 5 to 15%. Skin roughness is a measure of the rate of skin cell death. As skin cells die, a layer of dead cells forms on the skin surface, increasing its measured roughness. Therefore, skin which is classified as rough corresponds to increased skin cell death, compared to skin which is classified as smooth. Without wishing to be bound by theory, it is thought that poor hydration may especially be a factor leading to increased skin cell death, resulting in the above-described layer of dead cells forming on the skin surface, increasing its measured roughness.

Therefore, products (such as kits and compositions) and methods according to one or more (such as all) aspects of the present disclosure are preferably for reducing skin roughness. Yet more preferably, they are for reducing skin roughness while also reducing the depth and width of skin wrinkles, increasing skin hydration and reducing skin erythema.

As disclosed herein, it has been found to be possible to reduce skin roughness in a surprisingly short amount of time, for example within three months, or even within one month.

### DETAILED DESCRIPTION

### First Aspect of the Disclosure

Thus, according to a first aspect, the present disclosure provides a kit, comprising: a first composition for oral administration, comprising hyaluronic acid, one or more ceramides and manganese; and a second composition for oral administration, comprising retinol or a derivative thereof, one or more omega-3 fatty acids and one or more omega-6 fatty acids.

Surprisingly, the inventors have found that while some of these ingredients conventionally have been administered topically, they can be administered orally all together, i.e. via a different route of administration, and successfully reach the skin layers at sufficient levels to have a beneficial effect.

Thus, one or more of the following benefits may be provided in accordance with the present disclosure: increased hydration of the skin (for example, as measured using a Corneometer obtainable from Courage & Khazaka Electronic GmbH of Mathias-Brüggen-Straße 91, 50829 Köln, Germany); decreased transepidermal water loss (TEWL; for example, as measured using a Tewameter obtainable from Courage & Khazaka Electronic GmbH); increased skin elasticity (for example, as measured using a Cutometer obtainable from Courage & Khazaka Electronic GmbH); decreased erythema (for example, as measured using a Mexameter obtainable from Courage & Khazaka Electronic GmbH); decreased wrinkle depth (for example, as measured using a Visioscan obtainable from Courage & Khazaka Electronic GmbH); decreased wrinkle width (for example, as measured using a Visioscan obtainable from Courage & Khazaka Electronic GmbH); increased skin smoothness (for example, as measured using a Visioscan obtainable from Courage & Khazaka Electronic GmbH); decreased skin roughness (for example, as measured using a Visioscan obtainable from Courage & Khazaka Electronic GmbH); and decreased skin scaliness (for example, as measured using a Visioscan obtainable from Courage & Khazaka Electronic GmbH).

For example, TEWL (such as when measured using a Tewameter obtainable from Courage & Khazaka Electronic GmbH) may be reduced by at least 5%; by at least 10%; or by at least 15%. Suitably, increased hydration of the skin may be provided along with reduced TEWL, for the reason that the two may be correlated. For example, hydration (such as when measured using a Corneometer obtainable from Courage & Khazaka Electronic GmbH of Mathias-Brüggen-Straße 91, 50829 Koln, Germany) may be increased by at least 15%; for example, by from 30 to 90%.

Additionally or alternatively, elasticity (such as when measured using a Cutometer obtainable from Courage & Khazaka Electronic GmbH) may be increased by at least 5%, for example by from 5 to 15%.

Additionally or alternatively, skin roughness (such as when measured using a Visioscan obtainable from Courage & Khazaka Electronic GmbH) may be decreased by at least 5%, for example by from 5 to 15%.

Additionally or alternatively, erythema (such as when measured using a Mexameter obtainable from Courage & Khazaka Electronic GmbH) may be decreased by at least 5%, for example by 5 to 15%.

Additionally or alternatively, wrinkle depth (for example, as measured using a Visioscan obtainable from Courage & Khazaka Electronic GmbH) may be decreased by at least 10%, for example by between 10 and 30%. Additionally or alternatively, wrinkle width (for example, as measured using a Visioscan obtainable from Courage & Khazaka Electronic GmbH) may be decreased by at least 10%, for example by between 10 and 30%. Additionally or alternatively, and related to wrinkle depth and width, skin smoothness (for example, as measured using a Visioscan obtainable from Courage & Khazaka Electronic GmbH) may be increased by at least 5%, for example by between 5 and 15%.

Additionally or alternatively, benefits provided in accordance with the present disclosure may include an improvement in the composition of the skin sebum (for example, as measured using a Sebumeter obtainable from Courage & Khazaka Electronic GmbH). For example, said improvement may be or include an improvement in the % w/w in the skin sebum of one or more of: one or more triglycerides, cholesterol, one or more wax esters, one or more fatty acids and squalene in the sebum. Optionally, the % w/w of the one or more triglycerides in the sebum may change (for example, increase) by at least 1%. Optionally, said change (for example, increase) is capped at a change of 5%. Optionally, the % w/w of the cholesterol in the sebum may change (for example, increase) by at least 1%. Optionally, said change (for example, increase) is capped at a change of 5%. Optionally, the % w/w of the one or more wax esters in the sebum may change (for example, increase) by at least 1%. Optionally, said change (for example, increase) is capped at a change of 5%. Optionally, the % w/w of the one or more fatty acids in the sebum may change (for example, increase) by at least 1%. Optionally, said change (for example, increase) is capped at a change of 5%. Optionally, the % w/w of the squalene in the sebum may change (for example, increase) by at least 1%. Optionally, said change (for example, increase) is capped at a change of 5%. In accordance with the present disclosure, the occlusive barrier function of skin sebum, protecting the layers of the skin from water loss by evaporation from the skin surface, may be enhanced. Thus, by “an improvement in the composition of the skin sebum” may be meant an improvement in the occlusive barrier function of skin sebum, for example as evidenced by a reduction in TEWL of at least 5%; at least 10%; or at least 15%.

a. The First Composition

Optionally, the first composition may comprise at least 20 mg (e.g. at least 35 mg) hyaluronic acid; at least 1 mg (e.g. at least 3 mg) of the one or more ceramides; and at least 0.1 mg (e.g. at least 0.3 mg) manganese.

a. Hyaluronic Acid

Hyaluronic acid (“HA”; also known in the art as hyaluronan) may be obtained from Bioiberica, S.A.U, C/Antic Cami Tordera, 109-119, 08389 Palafolls, Barcelona, Spain. HA is a non-sulphated glycosaminoglycan (glycosaminoglycans are also known in the art as mucopolysaccharides). Thus, HA is a polymer formed of disaccharide monomers. HA forms part of the extracellular matrix of keratinocytes in the basal cell layer of the epidermis of human skin, and is thought to assist with keratinocyte proliferation. HA can also help to maintain a hydrated extracellular matrix, for the reason that HA is hygroscopic. Thus, HA can play a role in maintenance of healthy keratinocyte turnover while also enhancing tissue hydration. It can also have a protective role against solar UV radiation by acting as a free radical scavenger.

Suitably, the first composition may comprise hyaluronic acid having a molecular weight in a range of from 10 kDa to 5000 kDa (particularly 610 kDa to 790 kDa.).

The term “hyaluronic acid”, as used herein, encompasses the conjugate base, hyaluronate, and its salts; for example, sodium hyaluronate.

a. Ceramides

As disclosed herein, the first composition of the kit according to the first aspect of the disclosure may comprise at least 1 mg (e.g. at least 3 mg) of the one or more ceramides. Optionally, the first composition may comprise a Triticum aestivum (wheat) grain extract which in turn comprises the one or more ceramides (e.g., the wheat grain extract may comprise at least 1 mg or at least 3 mg of the one or more ceramides). Thus, the one or more ceramides may be or may comprise phytoceramides, referred to as such because they are derived from plant (phyto-) matter. Optionally, the first composition may comprise at least 100 mg (e.g. at least 125 mg) of the Triticum aestivum (wheat) grain extract comprising the one or more ceramides. Thus, although not necessarily, the first composition may comprise at least 1 mg (e.g. at least 3 mg) of the one or more ceramides when the first composition comprises at least 100 mg (e.g. at least 125 mg) of the Triticum aestivum (wheat) grain extract.

According to preferred embodiments, all of the ceramides present in the first composition of the kit according to the invention are wheat gain extract phytoceramides. Alternatively or additionally, all or a portion of the one or more ceramides are from a non-wheat grain extract source but may optionally be provided in the first composition in the wheat grain extract, for example pre-dissolved in the wheat grain extract.

A Triticum aestivum (wheat) grain extract comprising ceramides may be obtained as Lipowheat™ from Robertet Health and Beauty, 10 Avenue Yves-Emmanuel Baudoin, 06130, Grasse, France and may be incorporated in the first composition as an oil (which is liquid at 25° C. and 1 atm). Ceramides may be readily soluble in said oil, on the basis that ceramides are lipid molecules. A ceramide molecule comprises sphingosine and a fatty acid, linked by an amide bond. As lipids, ceramides have emollient properties. Ceramides provide an occlusive barrier in the stratum corneum, blocking water evaporation from the skin surface thus preventing undue water loss. This increases the level of hydration of the skin. Consequently, the ceramides of the first composition are thought to act in synergy with the hyaluronic acid of the first composition. While hyaluronic acid promotes a hydrated extracellular matrix in the epidermis, ceramides form an occlusive coating on the stratum corneum thus diminishing or preventing water loss via the skin surface.

The term “ceramides”, as used herein, encompasses all sources of ceramides and ceramide derivatives. Nonetheless, when the first composition comprises a Triticum aestivum (wheat) grain extract comprising the one or more ceramides, there may be additionally present in the Triticum aestivum (wheat) grain extract one or more glucosylceramides (also referred to as glucocerebrosides).

According to preferred embodiments, all of the ceramides present in the first composition of the kit according to the invention are wheat gain extract phytoceramides. Alternatively or additionally, all or a portion of the one or more ceramides are from a non-wheat grain extract source but may optionally be provided in the first composition in the wheat grain extract, for example pre-dissolved in the wheat grain extract.

a. Manganese

Manganese can act as a cofactor for several enzymes involved in the formation of a healthy extracellular matrix in the epidermis, such as, for example, glycosyltransferases. Meanwhile, manganese is an essential component of manganese superoxide dismutase (“MnSOD”) which is a key antioxidant enzyme in the mitochondria. Thus, manganese is important for the protection of cells from oxidative stress, such as that due to free radicals formed when the skin is exposed to UV-A and/or UV-B radiation. Since mitochondria use aerobic respiration to generate most of a cell's supply of adenosine triphosphate, their protection from oxidative stress is particularly important. Thus, manganese can act in synergy with hyaluronic acid to promote a healthy extracellular matrix in the epidermis, while also providing protection against oxidative stress, c.f. the function of HA as a free radical scavenger.

The term “manganese”, as used herein, encompasses all sources of manganese and manganese ions, particularly manganese (II) ions. A suitable source of manganese (II) ions is manganese gluconate. Manganese gluconate is obtainable from Jost Chemical Co., 8150 Lackland Rd. MO 63114 Saint Louis, USA. Thus, optionally, the second composition comprises manganese (II) salts which comprise or consist of manganese gluconate.

Where weights of manganese are specified in this disclosure, they are to be understood as total weights of manganese and/or manganese ions (for example, the total weight of Mn²⁺ ions) regardless of the salt form in which the ions may be provided. For example, a composition containing manganese gluconate as a source of manganese, and which is described as containing “0.4 mg manganese,” is to be understood as containing 0.4 mg Mn²⁺ ions; in this example, the 0.4 mg value is not inclusive of the weight of gluconate ions.

a. Further Glycosaminoglycans

Optionally, the first composition may further comprise one or more further glycosaminoglycans or derivatives thereof (an example of such a derivative being a sulphated glycosaminoglycan). Thus, the first composition may optionally comprise one or more chondroitins, such as chondroitin sulphate. The first composition may optionally comprise dermatan sulphate. The first composition may optionally comprise both chondroitin sulphate and dermatan sulphate.

Suitably, the first composition may optionally comprise at least 3 mg (e.g. at least 5 mg) of the one or more further glycosaminoglycans or derivatives thereof, for example, it may optionally comprise at least 3 mg (e.g. at least 5 mg) of chondroitin sulphate or dermatan sulphate or it may optionally comprise at least 3 mg (e.g. at least 5 mg) of chondroitin sulphate and dermatan sulphate, or it may optionally comprise at least 3 mg (e.g., at least 5 mg) of chondroitin sulphate and at least 3 mg (e.g., at least 5 mg) of dermatan sulphate.

For example the first composition may comprise at least 3 mg (e.g. at least 5 mg) chondroitin sulphate. Chondroitin sulphate is a sulphated glycosaminoglycan formed from a chain of alternating monomers of N-acetylgalactosamine and glucuronic acid. It is thought to exhibit anti-inflammatory activity. It is also thought to stimulate the synthesis of hyaluronic acid, thus when present, it may enhance the provision of hyaluronic acid by the first composition of the kit according to the first aspect of the present disclosure. It is also thought to promote the synthesis of proteoglycans. Chondroitin sulphate may decrease the catabolic activity of chondrocytes (cells responsible for cartilage formation) and may inhibit the synthesis of proteolytic enzymes. It may decrease the formation of nitric oxide, and other substances that contribute to damage of the extracellular matrix of the epidermis. Thus, to summarize, chondroitin sulphate may act in synergy with the hyaluronic acid of the first composition of the kit according to the first aspect of the present disclosure to promote healthy, hydrated skin.

Additionally or alternatively, the first composition may comprise dermatan sulphate, which is a glycosaminoglycan formed from disaccharide monomers that contain N-acetyl galactosamine and iduronic acid. Its repeating units are sulphated at various positions. For example the first composition may comprise at least 3 mg (e.g. at least 6 mg) dermatan sulphate. Dermatan sulphate may contribute, with hyaluronic acid and, if present, chondroitin sulphate, to the structure and function of the extracellular matrix of the epidermis, being able to bind to cells present there and mediate protein-protein interactions or enzymatic activity, thus being important to cellular responsiveness and the healthy development and homeostasis of the epidermis. It is to be understood that weights of glycosaminoglycans in this disclosure refer to the weight of the glycosaminoglycan molecule or glycosaminoglycan molecular ion, disregarding the weight of any counter-ion.

a. Collagen

Additionally or alternatively, the first composition may comprise collagen. The protein collagen contributes structurally to the extracellular matrix of the epidermis. Thus, when present in the first composition of the kit according to the first aspect of the present disclosure, it may act in synergy with hyaluronic acid and other components present, to promote the healthy development and homeostasis of the epidermis. For example, the first composition may comprise at least 3 mg (e.g. at least 5 mg) collagen.

a. The Second Composition

The second composition of the kit according to the first aspect of the disclosure may comprise at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids.

a. Retinol

Retinol, also called vitamin A1, is a lipid-soluble vitamin in the vitamin A family. Without wishing to be bound by theory, it is thought that a metabolite of retinol, retinoic acid, when used in accordance with the present disclosure, may help maintain the balance (homeostasis) within the epidermis. In particular, it is thought that administration of retinol may lead to an accumulation of hyaluronic acid in the extracellular matrix of keratinocytes in the basal cell layer of the epidermis of human skin. Thus, retinol may act in synergy to enhance the skin hydrating effect of hyaluronic acid described herein. Without wishing to be bound by theory, retinol, when used in accordance with the present disclosure, is also thought to stimulate the synthesis of collagen by fibroblasts, mediate the removal of degenerated elastin fibres (thus improving skin elasticity and reducing the appearance of skin wrinkles) and promote the formation of new blood vessels (angiogenesis).

Retinol derivatives include retinal (retinaldehyde), tretinoin (all-trans-retinoic acid), isotretinoin (13-cis-retinoic acid), and alitretinoin (9-cis-retinoic acid). Notably, retinol derivatives include esters of retinol, such as retinyl palmitate (the ester of retinol and palmitic acid).

Thus, optionally, the retinol or derivative thereof of the second composition may comprise or consist of one or more of: retinol, retinal (retinaldehyde), tretinoin (all-trans-retinoic acid), isotretinoin (13-cis-retinoic acid), alitretinoin (9-cis-retinoic acid) and one or more retinyl esters (such as retinyl palmitate). Optionally, the retinol or derivative thereof of the second composition may comprise or consist of one or more retinyl esters. For example, the retinol or a derivative thereof of the second composition may comprise or consist of retinyl palmitate.

Where reference is made herein to an amount in μg (such as at least 60 μg, e.g. at least 100 μg) of retinol or a derivative thereof, it will be appreciated that μg refers to μg retinol activity equivalent (RAE). 1 μg RAE is equal to 1 μg of retinol having the following structure:

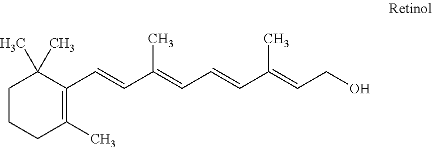 [SMILES: CC1=C(/C=C/C(C)=C/C=C/C(C)=C/CO)C(C)(C)CCC1]

Thus, μg RAE as a unit of measurement takes pure retinol as a standard and provides a way to measure retinol activity in vivo of retinol derivatives (such as retinyl esters, such as retinyl palmitate) as set out in Dietary Reference Intakes for Vitamin A, Vitamin K, Arsenic, Boron, Chromium, Copper, Iodine, Iron, Manganese, Molybdenum, Nickel, Silicon, Vanadium, and Zinc. US Food and Nutrition Board of the Institute of Medicine (2001) incorporated herein by reference as if set forth in its entirety.

a. Omega-3 Fatty Acids

Meanwhile, omega-3 fatty acids are polyunsaturated fatty acids (fatty acids that contain more than one double bond in their backbone) characterized by the presence of a double bond, three atoms away from the terminal methyl group in their chemical structure.

The known omega-3 fatty acids include:

| Common name | Lipid name | Chemical name |
|---|---|---|
| α-Linolenic acid (ALA) | 18:3 (n-3) | octadeca-9,12,15-trienoic acid |
| Stearidonic acid (SDA) | 18:4 (n-3) | octadeca-6,9,12,15-tetraenoic acid |
| Eicosatetraenoic acid (ETA) | 20:4 (n-3) | eicosa-8,11,14,17-tetraenoic acid |
| Eicosapentaenoic acid (EPA) | 20:5 (n-3) | eicosa-5,8,11,14,17-pentaenoic acid |
| Docosapentaenoic acid (DPA) | 22:5 (n-3) | docosa-7,10,13,16,19-pentaenoic acid |
| Docosahexaenoic acid (DHA) | 22:6 (n-3) | docosa-4,7,10,13,16,19-hexaenoic acid |

It is thought that omega-3 fatty acids, such as, for example, docosahexaenoic acid (DHA) and eicosapentaenoic acid (EPA), can act in synergy with ceramides in the stratum corneum to provide an enhanced occlusive barrier against water loss, thereby improving skin hydration. DHA and EPA are also thought to have a photoprotective effect, thus can mitigate solar UV-induced damage to skin cells. For example, they can protect against free radical-induced damage to cells, thereby complementing the action of manganese as disclosed herein, and hyaluronic acid as disclosed herein.

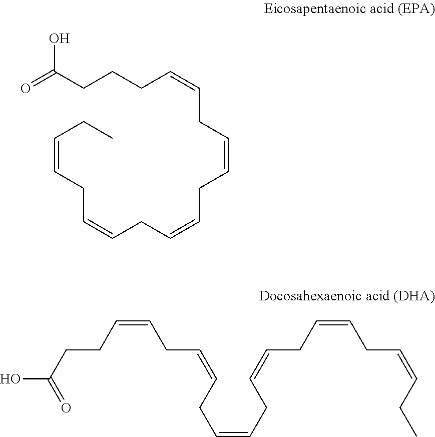 [SMILES: CC/C=C\C/C=C\C/C=C\C/C=C\C/C=C\CCCC(=O)O ; CC/C=C\C/C=C\C/C=C\C/C=C\C/C=C\C/C=C\CCC(=O)O]

Thus, optionally, the second composition comprises omega-3 fatty acids which comprise or consist of one or both of EPA and DHA. Optionally, the omega-3 fatty acids comprise or consist of both EPA and DHA. Optionally, the ratio of EPA to DHA is in a range of from 1:5 to 5:1 (e.g. 1:2 to 2:1).

As disclosed herein, the second composition of the kit according to the first aspect of the disclosure may comprise at least 300 mg (e.g. at least 500 mg) of one or more omega-3 fatty acids. However, optionally, the second composition may comprise a fish oil which in turn comprises the one or more omega-3 fatty acids, for example a fish oil which comprises one or both of EPA and DHA. Optionally, the first composition may comprise at least 700 mg (e.g. at least 900 mg) of the fish oil comprising the one or more omega-3 fatty acids. Thus, although not necessarily, the second composition may comprise at least 300 mg (e.g. at least 500 mg) of one or more omega-3 fatty acids when the second composition comprises at least 700 mg (e.g. at least 900 mg) of the fish oil.

Fish oil is oil derived from the tissues of oily fish (fish species with oils and/or fats in soft tissues and in the coelomic cavity around the gut) such as sardines, herring, anchovies, salmon, trout, tuna, swordfish and mackerel. As disclosed herein, fish oil comprises one or more omega-3 fatty acids, for example one or both of EPA and DHA. Said fish oil may be obtained from EPAX Norway AS, Aarsæthervegen 17, PO box 2047, NO-6028 Ålesund, Norway and is liquid at 25° C. and 1 atm. Omega-3 fatty acids may be readily soluble in said fish oil. The other components of fish oil may include one or more of: one or more triglycerides; one or more triacylglycerols; phosphatidylcholine; lysophosphatidylcholine; one or more phospholipids; one or more ethyl esters; one or more re-esterified triglycerides; and one or more re-esterified triacylglycerols.

a. Omega-6 Fatty Acids

Meanwhile, omega-6 fatty acids are polyunsaturated fatty acids that have a carbon-carbon double bond in the n-6 position, that is, the sixth bond, counting from the methyl end.

The known omega-6 fatty acids include:

| Common name | Lipid name | Chemical name |
|---|---|---|
| Linoleic acid (LA) | 18:2 (n-6) | all-cis-9,12-octadecadienoic acid |
| Gamma linoleic acid (GLA) | 18:3 (n-6) | all-cis-6,9,12-octadecatrienoic acid |
| Calendic acid | 18:3 (n-6) | 8E,10E,12Z-octadecatrienoic acid |
| Eicosadienoic acid | 20:2 (n-6) | all-cis-11,14-eicosadienoic acid |
| Dihomo-gamma-linolenic | 20:3 (n-6) | all-cis-8,11,14-eicosatrienoic acid |
| acid (DGLA) | | |
| Arachidonic acid (AA, ARA) | 20:4 (n-6) | all-cis-5,8,11,14-eicosatetraenoic acid |
| Docosadienoic acid | 22:2 (n-6) | all-cis-13,16-docosadienoic acid |
| Adrenic acid | 22:4 (n-6) | all-cis-7,10,13,16-docosatetraenoic acid |
| Osbond acid | 22:5 (n-6) | all-cis-4,7,10,13,16-docosapentaenoic acid |
| Tetracosatetraenoic acid | 24:4 (n-6) | all-cis-9,12,15,18-tetracosatetraenoic acid |
| Tetracosapentaenoic acid | 24:5 (n-6) | all-cis-6,9,12,15,18-tetracosapentaenoic |
| | | acid |

Omega-6 fatty acids (such as GLA) are thought to improve skin hydration and prevent or mitigate dermatitis through generation of anti-inflammatory metabolites (thought to include, for example, dihomo-GLA). Meanwhile, omega-6 fatty acids (such as GLA) may themselves contribute to the occlusive lipidic barrier in the stratum corneum, described herein with reference to ceramides and omega-3 fatty acids, to provide an enhanced occlusive barrier against water loss, thereby complementing the function of ceramides and omega-3 fatty acids and improving skin hydration.

Thus, optionally, the second composition comprises omega-6 fatty acids which comprise or consist of GLA.

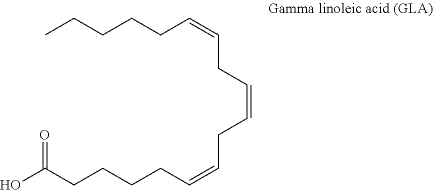 [SMILES: CCCCC/C=C\C/C=C\C/C=C\CCCCC(=O)O]

As disclosed herein, the second composition of the kit according to the first aspect of the disclosure may comprise at least 20 mg (e.g. at least 40 mg) of one or more omega-6 fatty acids. However, optionally, the second composition may comprise an Oenothera biennis (evening primrose) oil which in turn comprises the one or more omega-6 fatty acids, for example an Oenothera biennis oil which comprises GLA. Optionally, the first composition may comprise at least 300 mg (e.g. at least 400 mg) of the Oenothera biennis oil comprising the one or more omega-6 fatty acids. Thus, although not necessarily, the second composition may comprise at least 20 mg (e.g. at least 40 mg) of one or more omega-6 fatty acids when the second composition comprises at least 300 mg (e.g. at least 400 mg) of the Oenothera biennis oil.

Oenothera biennis (evening primrose) oil may be extracted from seeds of Oenothera biennis, the common evening primrose. Said oil may contain one or both of linoleic acid and gamma linoleic acid.

a. Capsule and Unit Dosage Formulations

The first composition may be provided in the form of at least one capsule. Additionally or alternatively, the second composition may be provided in the form of at least one capsule. Suitably, these capsules may be softgel capsules.

Optionally, when the first composition is provided in the form of at least one capsule, a single one of these capsules may contain at least 20 mg (e.g. at least 35 mg) hyaluronic acid; at least 1 mg (e.g. at least 3 mg) of the one or more ceramides; and at least 0.1 mg (e.g. at least 0.3 mg) manganese. Optionally, said single one of these capsules may contain at most 100 mg (e.g. at most 70 mg) hyaluronic acid; at most 15 mg (e.g. at most 6 mg) of the one or more ceramides; and at most 0.7 mg (e.g. at most 0.5 mg) manganese.

Optionally, when the first composition is provided in the form of at least one capsule, a single one of these capsules may contain at least 100 mg (e.g. at least 125 mg) of a Triticum aestivum (wheat) grain extract comprising the one or more ceramides. Optionally, said single one of these capsules may contain at most 250 mg (e.g. at most 200 mg) of a Triticum aestivum (wheat) grain extract comprising the one or more ceramides.

Optionally, when the first composition is provided in the form of at least one capsule, a single one of these capsules may contain at least 3 mg (e.g. at least 5 mg) of the one or more further glycosaminoglycans or derivatives thereof (e.g. chondroitin sulphate and/or dermatan sulphate). Optionally, it may contain at least 3 mg (e.g. at least 5 mg) collagen. Optionally, when the first composition is provided in the form of at least one capsule, a single one of these capsules may contain at most 20 mg (e.g. at most 15 mg) of the one or more further glycosaminoglycans or derivatives thereof (e.g. chondroitin sulphate and/or dermatan sulphate). Optionally, it may contain at most 20 mg (e.g. at least 10 mg) collagen.

Thus, optionally, when the first composition is provided in the form of at least one capsule, said capsule may contain about 43 mg hyaluronic acid; about 150 mg of a Triticum aestivum (wheat) grain extract comprising the one or more ceramides (containing, in turn, about 3 mg to about 6 mg ceramides); and about 0.4 mg manganese. Optionally, said capsule may further contain 6 mg chondroitin sulphate.

Optionally, when the second composition is provided in the form of at least one capsule, two of these capsules taken together may contain at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids. Optionally, said two of these capsules taken together may contain at most 200 μg (e.g. at most 150 μg) of retinol or a derivative thereof; at most 1000 mg (e.g. at most 750 mg) of one or more omega-3 fatty acids; and at most 80 mg (e.g. at most 70 mg) of one or more omega-6 fatty acids.

Optionally, when the second composition is provided in the form of at least one capsule, two of these capsules taken together may contain at least 700 mg (e.g. at least 900 mg) fish oil comprising the one or more omega-3 fatty acids. Optionally, said two of these capsules taken together may contain at most 2000 mg (e.g., at most 1500 mg) fish oil comprising the one or more omega-3 fatty acids.

Optionally, when the second composition is provided in the form of at least one capsule, two of these capsules taken together may contain at least 300 mg (e.g. at least 400 mg) of the Oenothera biennis (evening primrose) oil comprising the one or more omega-6 fatty acids. Optionally, said two of these capsules taken together may contain at most 1000 mg (e.g., at most 700 mg) Oenothera biennis oil comprising the one or more omega-6 fatty acids.

Thus, optionally, when the second composition is provided in the form of at least one capsule, two of these capsules taken together may contain about 120 μg RAE retinyl palmitate; about 1140 mg fish oil (containing, in turn, about 356 mg EPA and 238 mg DHA) and 594 mg Oenothera biennis (evening primrose) oil (containing, in turn, about 50 mg GLA).

The kit according to the first aspect of the present disclosure may comprise a unit dosage form, i.e. a form comprising a unit dosage of each of the first and second compositions of the kit. A unit dosage form may be or comprise one or more (for example, one to five, such as three) capsules, such as softgel capsules. For ease of swallowing, it may be preferred to limit the total weight of any one capsule to no more than 1000 mg, no more than 1500 mg or no more than 2000 mg. Optionally, the unit dosage form comprises one or more capsules packaged together in a container. For example, the kit may comprise a unit dosage form having three capsules, comprising (i) one softgel capsule comprising the first composition and (ii) two softgel capsules comprising the second composition, wherein, suitably, the three capsules are packaged together in a container such as a tear-open pod. Alternatively, they may be provided adjacent to each other in a multi-capsule blister pack which may optionally be marked, for example printed, to indicate that they are to be used together.

Said unit dosage form may contain at least 20 mg (e.g. at least 35 mg) hyaluronic acid; at least 1 mg (e.g. at least 3 mg) of the one or more ceramides; and at least 0.1 mg (e.g. at least 0.3 mg) manganese. Optionally, said unit dosage form may contain at most 100 mg (e.g. at most 70 mg) hyaluronic acid; at most 15 mg (e.g. at most 6 mg) of the one or more ceramides; and at most 0.7 mg (e.g. at most 0.5 mg) manganese.

Optionally, said unit dosage form may contain at least 100 mg (e.g. at least 125 mg) of a Triticum aestivum (wheat) grain extract comprising the one or more ceramides. Optionally, said unit dosage form may contain at most 250 mg (e.g. at most 200 mg) of a Triticum aestivum (wheat) grain extract comprising the one or more ceramides.

Said unit dosage form may contain at least 3 mg (e.g. at least 5 mg) of the one or more further glycosaminoglycans or derivatives thereof (e.g. chondroitin sulphate and/or dermatan sulphate). Optionally, it may contain at least 3 mg (e.g. at least 5 mg) collagen. The unit dosage form may contain at most 20 mg (e.g. at most 15 mg) of the one or more further glycosaminoglycans or derivatives thereof (e.g. chondroitin sulphate and/or dermatan sulphate). Optionally, it may contain at most 20 mg (e.g. at least 10 mg) collagen.

Thus, optionally, said unit dosage form may contain about 43 mg hyaluronic acid; about 150 mg of a Triticum aestivum (wheat) grain extract comprising the one or more ceramides (containing, in turn, about 3 mg to about 6 mg ceramides); and about 0.4 mg manganese. Optionally, said unit dosage form may further contain 6 mg chondroitin sulphate.

Said unit dosage form may contain at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids. Said unit dosage form may contain at most 200 μg (e.g. at most 150 μg) of retinol or a derivative thereof; at most 1000 mg (e.g. at most 750 mg) of one or more omega-3 fatty acids; and at most 80 mg (e.g. at most 70 mg) of one or more omega-6 fatty acids.

Said unit dosage form may contain at least 700 mg (e.g. at least 900 mg) fish oil comprising the one or more omega-3 fatty acids. Said unit dosage form may contain at most 2000 mg (e.g., at most 1500 mg) fish oil comprising the one or more omega-3 fatty acids.

Said unit dosage form may contain at least 300 mg (e.g. at least 400 mg) of the Oenothera biennis (evening primrose) oil comprising the one or more omega-6 fatty acids. Said unit dosage form may contain at most 1000 mg (e.g., at most 700 mg) Oenothera biennis oil comprising the one or more omega-6 fatty acids.

Thus, optionally, said unit dosage form may contain about 120 μg RAE retinyl palmitate; about 1140 mg fish oil (containing, in turn, about 356 mg EPA and 238 mg DHA) and 594 mg Oenothera biennis (evening primrose) oil (containing, in turn, about 50 mg GLA).

a. Excipients

Optionally, a composition disclosed herein (such as a composition of the kit according to the first aspect of the disclosure, or indeed a composition according to or as defined in any other aspect of the disclosure as disclosed herein) may comprise one or more lipid excipients. For example, a composition disclosed herein may be contained in a capsule as (or as a component of) the capsule filling, in which case the composition may comprise one or more lipid excipients such as one or more of rice bran oil, canola oil, vegetable oil, sunflower oil, olive oil, sesame oil, soybean oil, corn oil, peanut oil, palm oil, rapeseed oil, palm kernel oil, coconut oil, flaxseed oil or combinations thereof. Suitably, the lipid excipient is rice bran oil. For example, the first composition of the kit according to the first aspect of the disclosure may comprise rice bran oil.

Additionally or alternatively, when a composition disclosed herein is contained in a capsule, the composition may comprise one or more viscosity modifiers, such as one or more waxes, such as beeswax. Said viscosity modifier may be able to assist with preventing components from separating within the capsule, so as to obtain stability upon storage. For example, the first composition of the kit according to the first aspect of the disclosure may comprise beeswax.

Optionally, when a composition disclosed herein is contained in a capsule as (or as part of) the capsule filling, it may be encapsulated by a capsule shell. A capsule shell, such as a softgel capsule shell, may comprise one or more of gelatin (e.g. fish gelatin), beta carotene colour, caramel colour (E105a) and glycerol (E422). For example, the first composition of the kit according to the first aspect of the disclosure may be encapsulated by a capsule shell comprising gelatin (e.g. fish gelatin) and one or both of beta carotene colour and caramel colour (E105a). Meanwhile, the second composition of the kit according to the first aspect of the disclosure may be encapsulated by a capsule shell comprising gelatin (e.g. fish gelatin) and glycerol (E422).

### Second Aspect of the Disclosure

According to a second aspect, the present disclosure provides a composition for oral administration, comprising hyaluronic acid, one or more ceramides and manganese, which is formulated for use in a kit according to the first aspect of the present disclosure.

Optionally, the formulating the composition according to the second aspect of the disclosure for use in a kit according to the first aspect of the present disclosure comprises bringing together hyaluronic acid, one or more ceramides and manganese, in the amounts disclosed herein with reference to the first composition of the kit according to the first aspect of the disclosure. Optionally, said bringing together comprises encapsulation in a capsule for oral administration, for example a softgel capsule. Optionally, the source, identity and/or other properties of the hyaluronic acid, the one or more ceramides, and the manganese according to the second aspect of the invention may be as described in embodiments of the first aspect of the invention.

Optionally, the formulating the composition according to the second aspect of the disclosure for use in a kit according to the first aspect of the present disclosure comprises providing one or more further features of the first composition according to the first aspect of the present disclosure, for example adding one or more further glycosaminoglycans (e.g. chondroitin sulphate), in the amounts disclosed herein with reference to the first composition of the kit according to the first aspect of the disclosure.

### Third Aspect of the Disclosure

According to a third aspect, the present disclosure provides a composition for oral administration, comprising one or more omega-3 fatty acids, one or more omega-6 fatty acids and retinol, which is formulated for use in a kit according to the first aspect of the present disclosure.

Optionally, the formulating the composition according to the third aspect of the disclosure for use in a kit according to the first aspect of the present disclosure comprises bringing together one or more omega-3 fatty acids, one or more omega-6 fatty acids and retinol, in the amounts disclosed herein with reference to the second composition of the kit according to the first aspect of the disclosure. Optionally, said bringing together comprises encapsulation in a capsule for oral administration, for example a softgel capsule.

Optionally, the formulating the composition according to the third aspect of the disclosure for use in a kit according to the first aspect of the present disclosure comprises providing one or more further features of the second composition according to the first aspect of the present disclosure, for example providing the one or more omega-3 fatty acids in a fish oil and/or providing the one or more omega-6 fatty acids in an Oenothera biennis (evening primrose) oil.

Fourth Aspect of the disclosure

According to a fourth aspect, the present disclosure provides a composition for oral administration, comprising hyaluronic acid, one or more ceramides, manganese, retinol or a derivative thereof, one or more omega-3 fatty acids, and one or more omega-6 fatty acids.

It will be appreciated that the composition according to the fourth aspect of the disclosure may incorporate one or more features of the first composition of the kit according to the first aspect of the disclosure. Additionally or alternatively, the composition according to the fourth aspect of the disclosure may incorporate one or more features of the second composition of the kit according to the first aspect of the disclosure. To illustrate this point: the composition according to the fourth aspect of the present disclosure may comprise at least 20 mg (e.g. at least 35 mg) hyaluronic acid; at least 1 mg (e.g. at least 3 mg) of the one or more ceramides; at least 0.1 mg (e.g. at least 0.3 mg) manganese; at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids.

It will also be appreciated that the composition according to the fourth aspect of the disclosure may provide one or more of the benefits disclosed in relation to the kit according to the first aspect of the disclosure, for example the benefit(s) disclosed in relation to one or more of reduction in the depth and width of skin wrinkles; reduction in erythema; reduction in skin roughness; improvement in skin hydration; and improvement of the composition of the skin sebum.

Suitably, the composition according to the fourth aspect of the present disclosure may be contained in at least one capsule. Suitably, the at least one capsule may be at least one softgel capsule. It will be appreciated that the composition according to the fourth aspect of the present disclosure may be contained in at least one capsule which incorporates one or more features of the capsules disclosed herein with reference to the kit according to the first aspect of the disclosure. However, according to the fourth aspect of the disclosure, instead of having separate capsules for separate first and second compositions, the capsules (for example, one to five, such as three capsules, which may be softgel capsules) each contain the same composition, namely the composition according to the fourth aspect of the present disclosure. These capsules may be provided in a container, such as a tear-open pod. Alternatively, they may be provided adjacent to each other in a multi-capsule blister pack which may optionally be marked, for example printed, to indicate that they are to be used together.

The composition according to the fourth aspect of the present disclosure may be provided in unit dosage form. It will be appreciated that the unit dosage form of the composition according to the fourth aspect of the present disclosure may incorporate one or more features of the unit dosage form disclosed herein with reference to the kit according to the first aspect of the disclosure. However, instead of having separate capsules for separate first and second compositions, this unit dosage form may comprise one or more (for example, one to five, such as three) capsules (e.g. softgel capsules) each containing the same composition, namely the composition according to the fourth aspect of the present disclosure. These capsules may be provided in a container, such as a tear-open pod. Alternatively, they may be provided adjacent to each other in a multi-capsule blister pack which may optionally be marked, for example printed, to indicate that they are to be used together.

### Fifth Aspect of the Disclosure

According to a fifth aspect, the present disclosure provides a method (e.g., a cosmetic method) of: increasing skin hydration; and/or reducing the depth and width of skin wrinkles, the method comprising administering the first and second compositions of the kit according to the first aspect of the present disclosure or the composition according to the fourth aspect of the present disclosure to a human subject.

Optionally, in the method according to the fifth aspect of the present disclosure, the first composition of the kit according to the first aspect of the disclosure may be administered together with food. Optionally, the second composition may be administered with food. Optionally, both the first composition and the second composition may be administered with food.

Optionally, in the method according to the fifth aspect of the present disclosure, the composition according to the fourth aspect of the disclosure may be administered with food.

It will be appreciated that the method according to the fifth aspect of the disclosure may provide one or more of the benefits disclosed in relation to the kit according to the first aspect of the disclosure, for example the benefit(s) disclosed in relation to one or more of reduction in the depth and width of skin wrinkles; reduction in erythema; reduction in skin roughness; improvement in skin hydration; and improvement of the composition of the skin sebum. Notably, as disclosed herein, it has been found to be possible to treat or mitigate skin dryness (i.e., increase skin hydration) in a surprisingly short amount of time. It has also been found to be possible to reduce the width and depth of skin wrinkles in a surprisingly short amount of time. Thus, the method according to the fifth aspect of the disclosure may comprise administering the first and second compositions of the kit according to the first aspect of the present disclosure or the composition according to the fourth aspect of the present disclosure to a human subject for up to 3 months, for up to 2 months, or for up to 1 month only. According to certain embodiments, treatment for such a length of time produces an improvement in the skin of at least an extent noted elsewhere in this disclosure. For example, TEWL (such as when measured using a Tewameter obtainable from Courage & Khazaka Electronic GmbH) may be reduced by at least 5%; by at least 10%; or by at least 15%. Suitably, increased hydration of the skin may be provided along with reduced TEWL, for the reason that the two may be correlated. For example, hydration (such as when measured using a Corneometer obtainable from Courage & Khazaka Electronic GmbH of Mathias-Brüggen-Straße 91, 50829 Koln, Germany) may be increased by at least 15%; for example, by from 30 to 90%. Additionally or alternatively, elasticity (such as when measured using a Cutometer obtainable from Courage & Khazaka Electronic GmbH) may be increased by at least 5%, for example by from 5 to 15%. Additionally or alternatively, erythema (such as when measured using a Mexameter obtainable from Courage & Khazaka Electronic GmbH) may be decreased by at least 5%, for example by 5 to 15%. Additionally or alternatively, wrinkle depth (for example, as measured using a Visioscan obtainable from Courage & Khazaka Electronic GmbH) may be decreased by at least 10%, for example by between 10 and 30%. Additionally or alternatively, and related to wrinkle depth, skin smoothness (for example, as measured using a Visioscan obtainable from Courage & Khazaka Electronic GmbH) may be increased by at least 5%, for example by between 5 and 15%.

The method according to the fifth aspect of the disclosure may comprise administering the first composition of the kit according to the first aspect of the present disclosure in the form of at least one capsule. For example, the method may comprise administering one to five (e.g. one) capsules containing the first composition daily. Said capsule may contain at least 20 mg (e.g. at least 35 mg) hyaluronic acid; at least 1 mg (e.g. at least 3 mg) of the one or more ceramides; and at least 0.1 mg (e.g. at least 0.3 mg) manganese. Optionally, said capsule may contain at most 100 mg (e.g. at most 70 mg) hyaluronic acid; at most 15 mg (e.g. at most 6 mg) of the one or more ceramides; and at most 0.7 mg (e.g. at most 0.5 mg) manganese. Optionally, said capsule may contain at least 100 mg (e.g. at least 125 mg) of a Triticum aestivum (wheat) grain extract comprising the one or more ceramides. Optionally, said capsule may contain at most 250 mg (e.g. at most 200 mg) of a Triticum aestivum (wheat) grain extract comprising the one or more ceramides. Optionally, said capsule may contain at least 3 mg (e.g. at least 5 mg) of the one or more further glycosaminoglycans or derivatives thereof (e.g. chondroitin sulphate and/or dermatan sulphate). Optionally, it may contain at least 3 mg (e.g. at least 5 mg) collagen. Optionally, said capsule may contain at most 20 mg (e.g. at most 15 mg) of the one or more further glycosaminoglycans or derivatives thereof (e.g. chondroitin sulphate and/or dermatan sulphate). Optionally, it may contain at most 20 mg (e.g. at least 10 mg) collagen.

Additionally or alternatively, the method according to the fifth aspect of the disclosure may comprise administering the second composition of the kit according to the first aspect of the present disclosure in the form of at least one capsule. For example, the method may comprise administering one to five (e.g. two) capsules containing the second composition daily. Optionally, two capsules containing the second composition, taken together, may contain at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids. Optionally, said two capsules, taken together, may contain at most 200 μg (e.g. at most 150 μg) of retinol or a derivative thereof; at most 1000 mg (e.g. at most 750 mg) of one or more omega-3 fatty acids; and at most 80 mg (e.g. at most 70 mg) of one or more omega-6 fatty acids.

Thus, the method according to the fifth aspect of the disclosure may comprise administering one to five (e.g. one) capsules containing the first composition of the kit according to the first aspect of the disclosure daily, and also administering one to five (e.g. two) capsules containing the second composition of the kit according to the first aspect of the disclosure daily. Optionally, said daily administration may be repeated for one to three months, such as for three months, two months, or one month. Optionally, said daily administration may be repeated for up to one month only.

Thus, suitably, the method according to the fifth aspect of the disclosure may comprise administering one capsule containing the first composition of the kit according to the first aspect of the disclosure daily, and also administering two capsules containing the second composition of the kit according to the first aspect of the disclosure daily. Optionally, this daily administration may be repeated for three months, two months, or one month. Optionally, this daily administration may be repeated for up to one month only.

As disclosed herein, the kit according to the first aspect of the present disclosure may comprise a unit dosage form, i.e. a form comprising a unit dosage of each of the first and second compositions of the kit. Thus, the method according to the fifth aspect of the present disclosure may comprise administering said unit dosage form. Optionally, the method according to the fifth aspect of the present disclosure may comprise administering said unit dosage form one to five times (e.g. once) daily, such as one to five times (e.g. once) daily for one to three months, for example for three months, for two months, or for one month. Thus, optionally, the method according to the fifth aspect of the present disclosure may comprise administering said unit dosage form once daily for up to one month only.

Alternatively, the method according to the fifth aspect of the disclosure may comprise administering the composition according to the fourth aspect of the present disclosure daily. Optionally, the method may comprise administering a unit dosage form of the composition according to the fourth aspect of the present disclosure, as disclosed herein, one to five times (e.g. once) daily. Optionally, said daily administration may be repeated for one to three months, such as for three months, two months, or one month. Thus, optionally, the method according to the fifth aspect of the present disclosure may comprise administering said unit dosage form once daily for up to one month only.

It will be appreciated that the kit according to the first aspect of the disclosure may comprise a leaflet, setting out instructions for carrying out a method according to the fifth aspect of the present disclosure.

### Sixth Aspect of the Disclosure

According to a sixth aspect, the present disclosure provides a method, comprising: (i) orally administering to a human subject at least a first composition, comprising hyaluronic acid, one or more ceramides and manganese; and (ii) orally administering to the human subject at least a second composition, comprising one or more omega-3 fatty acids, one or more omega-6 fatty acids and retinol; wherein steps (i) and (ii) are each repeated at least once per day.

Optionally, in the method according to the sixth aspect of the present disclosure, the first and second compositions may be the first and second compositions of the kit according to the first aspect of the disclosure. Thus, the first and second compositions may incorporate any of the features of the first and second compositions of the kit according to the first aspect of the disclosure, as disclosed herein.

Optionally, in the method according to the sixth aspect of the present disclosure, the first composition may be administered with food. Optionally, the second composition may be administered with food. Optionally, both the first composition and the second composition may be administered with food.

It will also be appreciated that the method according to the sixth aspect of the disclosure may provide one or more of the benefits disclosed in relation to the kit according to the first aspect of the disclosure, for example the benefit(s) disclosed in relation to one or more of reduction in the depth and width of skin wrinkles; reduction in erythema; reduction in skin roughness; improvement in skin hydration; and improvement of the composition of the skin sebum. Notably, as disclosed herein, it has been found to be possible to treat or mitigate skin dryness and to reduce the depth and width of skin wrinkles in a surprisingly short amount of time. Thus, the method according to the sixth aspect of the disclosure may comprise administering the first and second compositions to a human subject for one to three months, for example for three months, for two months, or for one month, such as, for example, for up to 1 month only.

The method according to the sixth aspect of the disclosure may comprise administering the first composition in the form of at least one capsule. For example, the method may comprise administering a capsule containing the first composition daily. Said capsule may contain at least 20 mg (e.g. at least 35 mg) hyaluronic acid; at least 1 mg (e.g. at least 3 mg) of the one or more ceramides; and at least 0.1 mg (e.g. at least 0.3 mg) manganese. Optionally, said capsule may contain at most 100 mg (e.g. at most 70 mg) hyaluronic acid; at most 15 mg (e.g. at most 6 mg) of the one or more ceramides; and at most 0.7 mg (e.g. at most 0.5 mg) manganese. Optionally, said capsule may contain at least 100 mg (e.g. at least 125 mg) of a Triticum aestivum (wheat) grain extract comprising the one or more ceramides. Optionally, said capsule may contain at most 250 mg (e.g. at most 200 mg) of a Triticum aestivum (wheat) grain extract comprising the one or more ceramides. Optionally, said capsule may contain at least 3 mg (e.g. at least 5 mg) of the one or more further glycosaminoglycans or derivatives thereof (e.g. chondroitin sulphate and/or dermatan sulphate). Optionally, it may contain at least 3 mg (e.g. at least 5 mg) collagen. Optionally, said capsule may contain at most 20 mg (e.g. at most 15 mg) of the one or more further glycosaminoglycans or derivatives thereof (e.g. chondroitin sulphate and/or dermatan sulphate). Optionally, it may contain at most 20 mg (e.g. at least 10 mg) collagen. The method according to the sixth aspect of the disclosure may comprise administering from one to five (e.g. one) capsules containing the first composition daily.

Additionally or alternatively, the method according to the sixth aspect of the disclosure may comprise administering the second composition in the form of at least one capsule. For example, the method may comprise administering two capsules containing the second composition daily. Optionally, two capsules containing the second composition, taken together, may contain at least 60 μg (e.g. at least 100 μg) of retinol or a derivative thereof; at least 300 mg (e.g. at least 500 mg) of one or more omega-3 fatty acids; and at least 20 mg (e.g. at least 40 mg) of one or more omega-6 fatty acids. Optionally, said two capsules, taken together, may contain at most 200 μg (e.g. at most 150 μg) of retinol or a derivative thereof; at most 1000 mg (e.g. at most 750 mg) of one or more omega-3 fatty acids; and at most 80 mg (e.g. at most 70 mg) of one or more omega-6 fatty acids. The method according to the sixth aspect of the disclosure may comprise administering from one to five (e.g. two) capsules containing the second composition daily.

Thus, suitably, the method according to the sixth aspect of the disclosure may comprise administering one capsule containing the first composition daily, and also administering two capsules containing the second composition daily. Optionally, this daily administration may be repeated for one to three months, for example for three months, two months, or one month. Optionally, this daily administration may be repeated for up to one month only.

As disclosed herein, the kit according to the first aspect of the present disclosure may comprise a unit dosage form, i.e. a form comprising a unit dosage of each of the first and second compositions of the kit. Thus, the method according to the sixth aspect of the present disclosure may comprise administering said unit dosage form. Optionally, the method according to the sixth aspect of the present disclosure may comprise administering said unit dosage form one to five times (e.g. once) daily, such as one to five times (e.g. once) daily for one to three months (e.g. for three months, for two months, or for one month). Thus, optionally, the method according to the sixth aspect of the present disclosure may comprise administering said unit dosage form once daily for up to one month only.

The kit according to the first aspect of the disclosure may comprise a leaflet, setting out instructions for carrying out a method according to the sixth aspect of the present disclosure.

### Seventh Aspect of the Disclosure

According to a seventh aspect, the present disclosure provides a method of making the kit according to the first aspect of the disclosure, comprising placing the first composition and the second composition together in a suitable container, such as a tear-open pod. Alternatively, they may be provided adjacent to each other in a multi-capsule blister pack which may optionally be marked, for example printed, to indicate that they are to be used together. The compositions may optionally comprise one or more features disclosed herein as optional features in respect of one or more other aspects of the invention.

### Eighth Aspect of the Disclosure

According to an eighth aspect, the present disclosure provides a method of making the kit according to the first aspect of the disclosure, comprising a step of (i) a formulating said first composition; and/or a step of (ii) formulating said second composition. The compositions may optionally comprise one or more features disclosed herein as optional features in respect of one or more other aspects of the invention.

### Ninth Aspect of the Disclosure

According to an ninth aspect, the present disclosure provides a kit according to the first aspect of the disclosure or a composition according to the second aspect of the disclosure, for use as a medicament. Suitably, said kit or composition may be provided for use in the treatment of one or more of the following conditions: dermatitis, eczema, psoriasis, and xeroderma (also known as xerosis). The kit or composition for use according to the ninth aspect of the disclosure may optionally comprise one or more features disclosed herein as optional features in respect of one or more other aspects of the disclosure; it may provide one or more of the benefits disclosed herein in relation to one or more other aspects of the disclosure, for example the benefit(s) disclosed in relation to the composition of the skin sebum.

### Tenth Aspect of the Disclosure

According to a tenth aspect, the present disclosure provides a method of treatment, such as treatment of one or more of dermatitis, eczema, psoriasis, or xeroderma (also known as xerosis). The method of treatment according to the tenth aspect of the disclosure may optionally comprise one or more features disclosed herein as optional features in respect of one or more other aspects of the invention. Thus, said method of treatment may comprise one or more of the steps described herein with reference to the fifth aspect of the disclosure. Additionally or alternatively, said method of treatment may comprise one or more of the steps described herein with reference to the sixth aspect of the disclosure. The method according to the tenth aspect of the disclosure may provide one or more of the benefits disclosed herein in relation to one or more other aspects of the invention, for example the benefit(s) disclosed in relation to the composition of the skin sebum.

### Optional Features

It will of course be appreciated that features and benefits described in relation to one aspect of the present disclosure may be incorporated into other aspects of the present disclosure.

### EXAMPLES

Two studies are cited below for their review of the effect of dietary supplementation on skin parameters.

Dietary supplementation of gamma-linolenic acid improves skin parameters in subjects with dry skin and mild atopic dermatitis, Kawamura, A., et al., J. Oleo. Sci., 2011, 60 (12), 597-607 (incorporated herein by reference as if set forth in its entirety) discloses a study to assess the effect of orally administered gamma linoleic acid (GLA) on human subjects with dry skin and mild to moderate atopic dermatitis. 130 volunteers were recruited and were divided into two groups (one placebo, one active). Measurements were taken before treatment (week 0) and at 4, 8, 12 and 16 weeks after treatment to determine skin hydration and TEWL Blood tests, urinalysis, a survey of subjective symptoms and examination by a physician were also performed. 200 mg/day GLA was administered to each subject in the active group. The orally administered GLA was found to reduce TEWL Statistically significant efficacy was demonstrated, especially in subjects with high levels of pro-inflammatory substances.

Supplementation with Eskimo® Skin Care improves skin elasticity in women. A pilot study, Segger, D., et al., J. Derm. Treatment, 2008, 19 (5), 279-283 (incorporated herein by reference as if set forth in its entirety) discloses a study to assess the effect of supplementation with fish oil on skin elasticity, TEWL and skin roughness in healthy women. 24 female volunteers aged 40-60 years were recruited and were divided into two groups (one placebo, one active). 10 ml/day of Eskimo® Skin Care was administered, containing 70% w/w fish oil, 20% w/w evening primrose oil, 10% w/w canola oil, and 40 IU/ml vitamin D. The fish oil contains 12.7% w/w EPA, 8.1% w/w DHA and 2.1% w/w GLA. After a study duration of 12 weeks, there was found to be a 10% increase in skin elasticity of the active group.

### Example 1

A cachet containing 200 mg hyaluronic acid (HA) was administered orally, once daily, to 30 subjects for 28 days. The hyaluronic acid administered in this manner consisted of hyaluronic acid molecules having molecular weights in a range of from 50 to 2200 kDa. The hydration, TEWL and wrinkle depth of the skin of the cheek of each subject was measured on the first day of the 28-day study and on the last day of the 28-day study. A Courage+Khazaka Dual 580 Multi Probe Adapter System fitted with a Corneometer™ CM 825 probe (obtainable from Courage & Khazaka Electronic GmbH of Mathias-Brüggen-Straße 91, 50829 Köln, Germany) was used to measure the surface hydration of the subjects' skin. A Courage+Khazaka Dual 580 Multi Probe Adapter System fitted with a Tewameter™ 300 probe was used to measure the TEWL of the subjects' skin. A PRIMOS 3D™ optical skin measurement device was used to measure the depth and width of wrinkles in the subjects' skin. The average percentage change in each of the parameters measured from the first day of the 28-day study to the last day of the 28-day study was calculated. The results are shown in Table 1.

TABLE 1
Average percentage change in each of the parameters measured

| | Average percentage change over the |
|---|---|
| Parameter | course of the 28-day study |
| Wrinkle depth | −18.8% |
| Skin surface hydration | +10.6% |
| TEWL | −3.9% |

### Example 2

350 mg wheat grain extract oil (comprising 2 to 4% phytoceramides by weight, relative to the total weight of the wheat grain extract oil) was administered orally, once daily, to 25 subjects for three months. The hydration of the skin of the cheek of each subject was measured on the first day of the three-month study and on the last day of the three-month study. The average percentage change in the hydration of the skin of the cheek from the first day of the three-month study to the last day of the three-month study was calculated and was found to be +26.0%.

### Example 3

A softgel capsule was prepared having the shell composition of Table 2a and the filling composition of Table 2b.

TABLE 2a
Shell composition of softgel capsule

| | Quantity in milligrams |
|---|---|
| Shell component | (mg) |
| Fish Gelatin | 223.38 |
| Glycerol | 143.56 |
| Betacarotene (30% by weight in sunflower oil) | 8.6 |
| Caramel colour E150a | 8.6 |

The total weight of the shell of Table 2a is 385.00 mg.

TABLE 2b
Filling composition of softgel capsule

| Filling component | Quantity in milligrams (mg) |
|---|---|
| Rice bran oil | 500.00 |
| Wheat grain extract oil | 150.00 |
| Hyaluronic acid (consisting of hyaluronic | 100.00 |
| acid molecules having molecular weights in | |
| a range of from 50 to 2200 kDa) | |
| Beeswax | 30.36 |
| Manganese gluconate | 3.64 |

The total weight of the filling of Table 2b is 784.00 mg.

The softgel capsule was administered twice daily to 12 subjects for 12 weeks. The age of the subjects ranged from 30 to 61 years. The elasticity, hydration and transepidermal water loss (TEWL) of the skin surrounding the eye, the skin of the cheek, the skin of the neck, and the skin of the forearm of each subject was measured on the first day of the 12-week study and on the last day of the 12-week study. The depth and width of wrinkles in those areas of the subjects' skin was also measured on the first day of the 12-week study and on the last day of the 12-week study. The roughness of the skin of the cheek was also measured on the first day of the 12-week study and on the last day of the 12-week study. A Courage+Khazaka Dual 580 Multi Probe Adapter System fitted with a Cutometer™ MPA 580 probe was used to measure the elasticity of the subjects' skin. A Courage+Khazaka Dual 580 Multi Probe Adapter System fitted with a Corneometer™ CM 825 probe was used to measure the surface hydration of the subjects' skin. A Courage+Khazaka Dual 580 Multi Probe Adapter System fitted with a Tewameter™ TM 300 probe was used to measure the TEWL of the subjects' skin. A Courage+Khazaka Visoscan™ VC98 was used to measure the depth and width of wrinkles in the subjects' skin. A Visioscan® VC 98 was used to measure skin roughness of the cheek. The average percentage change in each of the parameters measured from the first day of the twelve week study to the last day of the twelve week study was calculated. The results are shown in Table 3.

TABLE 3
Average percentage change in each of the parameters measured

| | Skin | Average percentage change over the |
|---|---|---|
| Parameter | region | course of the 12-week study |
| Elasticity | Eye | −7% |
| | Cheek | −6% |
| | Neck | −3% |
| | Arm | −2% |
| Wrinkle width | Eye | −20% |
| | Cheek | +16% |
| | Neck | −19% |
| | Arm | +3% |
| Wrinkle depth | Eye | −28% |
| | Cheek | +18% |
| | Neck | −25% |
| | Arm | +9% |
| Skin surface | Eye | +31% |
| hydration | Cheek | +64% |
| | Neck | +38% |
| | Arm | +11% |
| TEWL | Eye | +18% |
| | Cheek | −20% |
| | Neck | −17% |
| | Arm | −21% |
| Roughness | Cheek | +29% |

The increase in the level of hydration of the skin of the cheek resulting from administration of the softgel capsule of Example 3 demonstrates a greater than additive effect (+64.0%) compared to the sum of the increases seen for HA alone in Example 1 (+10.6%) and phytoceramides alone in Example 2 (+26.0%). This suggests that, when administered in combination, HA and phytoceramides act in synergy to increase the level of hydration of the skin.

### Example 4

A softgel capsule was prepared having the composition of Tables 4 to 6.

TABLE 4
Ingredients and their amounts in a single softgel capsule

| | Ingredient | Weight | |
|---|---|---|---|
| Filling | Fish oil | 570 | mg |
| | d alpha tocopherol (E306) | 1 | mg |
| | Oenothera biennis oil | 297 | mg |
| | Retinyl palmitate | 60 μg RAE* | |
| Shell | Fish gelatin | 267.99 | mg |
| | Glycerol (E422) | 107.598 | mg |

*μg RAE is μg retinol activity equivalent as defined herein.

TABLE 5
Active ingredients of fish oil in a single softgel capsule

| Ingredient | Weight (mg) |
|---|---|
| Eicosapentaenoic acid (EPA) | 178 |
| Docosahexaenoic acid (DHA) | 119 |

TABLE 6
Active ingredient of Oenothera biennis
oil in a single softgel capsule

| Ingredient | Weight (mg) |
|---|---|
| Gamma linoleic acid (GLA) | 25 |

Two of the softgel capsules were administered daily to 9 subjects for 12 weeks. The age of the subjects ranged from 22 to 51 years, with an average age of 34. The hydration and wrinkle depth and width of the skin of the cheek of each subject, was measured on the first day of the 12-week study and on the last day of the 12-week study. The mean percentage change in each of the parameters measured from the first day of the twelve week study to the last day of the twelve week study was calculated. Non-invasive probe devices and facial imaging equipment were used, obtainable from Courage & Khazaka Electronic GmbH of Mathias-Brüggen-Straße 91, 50829 Koln, Germany:
Corneometer® CM825 (measuring hydration)
Visioscan® VC 98 (measuring wrinkle depth and width)

Confirmatory full face photos were taken with a VISIA® facial imaging machine obtainable from Canfield Scientific of 4 Wood Hollow Road, Parsippany, NJ 07054, USA.

The results are shown in Table 7.

TABLE 7
Results of Example 4

| | Area of face | Mean % |
|---|---|---|
| Parameter Measured | measured | improvement |
| Hydration | Cheek | 40% |
| Wrinkle Depth | Cheek | 12% |
| Wrinkle Width | Cheek | 5% |

### Example 5

Example 5 assessed the effects of products and methods according to the present disclosure on the following skin parameters in a group of human volunteers: wrinkle depth, wrinkle width, skin roughness, hydration, and erythema.

### Subjects and Study Design

6 healthy female participants of ages 29-44 fulfilling the specification for “dry” skin (for the purposes of the study, a participant will be classed as having “dry” skin if the skin in question displays a Corneometer value of less than 30) were recruited.

TABLE 8
Individual Corneometer ® CM825 (hydration)
assessment scores for participants

| Participant | Hydration score (Cheek area) |
|---|---|
| AD019RD18 | 17.9 |
| AM014RD18 | 27.8 |
| KA013RD18 | 28.4 |
| LS032RD18 | 18.0 |
| PP026RD18 | 6.9 |
| GC011RD18 | 25.4 |

All participants attended their appointments make-up free. All participants washed their faces with a micro-fibre chemical- and soap-free cleansing mitt at the start of the appointment. Acclimatization was required for each participant for 20 minutes in a room at 21° C. before any measurements were taken.

### Equipment

Non-invasive probe devices and facial imaging equipment were used, obtainable from Courage & Khazaka Electronic GmbH of Mathias-Brüggen-Straße 91, 50829 Koln, Germany:
Corneometer® CM825 (measuring hydration)
Tewameter® TM 300 (measuring Transepidermal Water Loss)
Mexameter® MX18 (measuring erythema and melanin)
Visioscan® VC 98 (measuring wrinkle depth, wrinkle width, skin roughness)

The cheek area was chosen as the area of the skin to be investigated. The cheek is typically the largest single area of the skin exposed to the surrounding environment and weather. It may therefore be the most difficult area of skin for which to improve hydration and reduce wrinkle depth and width.

Full face photos were taken with a VISIA® facial imaging machine obtainable from Canfield Scientific of 4 Wood Hollow Road, Parsippany, NJ 07054, USA.

### Inclusion Criteria for Study Participants

The participant is able to read and understand the Informed Consent Form (ICF) and understand study procedures.
The participant has signed the Informed Consent Form (ICF).
Fitzpatrick classification I, II, Ill, IV, V to be included.
Healthy male or female participants aged 25 and upwards.
The participant is a non-smoker who has not used nicotine or nicotine-containing products for at least 1 month prior to screening and for the duration of the study.
Currently not using any prescribed topical medications.
Currently not using any prescribed internal medications, other than the oral contraceptive pill (needs to have been on stable treatment for 3 months prior to screening and that no changes will be made to this treatment).
Willing to stop receiving any laser/light therapy facial treatments from screening and for the duration of the study.
Willing to stop taking other forms of supplementation from screening and for the duration of the study.
Willing to avoid adding in anything new to their topical skincare routine for the duration of the study e.g. booking courses of facials or anti-ageing treatments, Botox, fillers, dermabrasion, any therapeutic treatments that will affect skin.
Willing to avoid excessive amounts of UV light for the duration of the study.
The participant is healthy on the basis of medical history.

### Exclusion Criteria for Study Participants

Volunteers taking any prescription medication (topical and internal).
Volunteers currently taking regular prescribed concurrent medications or hormones (other than oral contraception, which is of a stable dose for at least the last 3 months).
   EXEMPT from the medications clause are: vaccinations or using over-the-counter medications (except for occasional over the counter analgesics (a maximum of 2 g paracetamol per day is permissible), cold remedies and anti-histamines.
Currently participating in another study that involves taking medications or food supplements or applying topical anti-ageing products.
Serious seasonal allergies (mild seasonal allergies treated with cetirizine are allowed).
Pregnant, breastfeeding or attempting to become pregnant.
Underlying diseases or other dermatologic conditions that require the use of topical or systemic therapy such as, but not limited to, atopic dermatitis, perioral dermatitis or rosacea.
Use of tanning booths or other light devices within 2 weeks prior to or planned used during the study.
History of active uncontrolled gastrointestinal diseases or disorders including: Crohns, Ulcerative colitis, IBD or IBS.
Immunosuppression (Chronic steroid use, Active chemotherapy, Active radiotherapy, HIV or any other disorder resulting in a compromise in participant's immune status).
Allergic to fish products.
Volunteers with damaged skin at or in close proximity to test sites (e.g. sunburn, tattoos, scars, excessive hair, non-removable piercings, or other disfigurations on and around facial areas.)
Volunteers using any retinoid-based cosmeceuticals.
Volunteers who have had any facial procedures including facials, chemical peels, dermabrasion in the 2 weeks prior to and during the survey.

### Survey Period

### 12-Week (i.e., Approx. 3-Month) Study.

First check in appointment at week 1.
Final appointment week 12.

All participants continued with their existing skincare routine only, for the duration of the study, save that products containing retinol, hyaluronic acid, peptides or ceramides were discontinued. Participants were forbidden to take any other food supplements for the duration of the study unless medically prescribed.

### Methods and Materials

The oral supplementation provided to each participant consisted of a plain labelled box containing a 28-day supply of tear-open pods. Each pod contained one softgel capsule containing the first composition of the kit according to the first aspect of the present disclosure and two softgel capsules containing the second composition of the kit according to the first aspect of the present disclosure.

One tear-open pod was taken daily with food. Enough supply was given to participants for the duration of the 12 weeks.

TABLE 9
Ingredients and their amounts in a single softgel
capsule containing the first composition

| | | Weight |
|---|---|---|
| | Ingredient | (mg) |
| Filling | Hyaluronic acid (consisting of hyaluronic acid | 43 |
| | molecules having molecular weights in a range of | |
| | from 610 kDa to 790 kDa) | |
| | Triticum aestivum (wheat) grain extract | 149.7 |
| | Rosemary leaf extract in sunflower oil | 0.3 |
| | Manganese (from manganese gluconate) | 0.4 |
| | Chondroitin sulphate | 6 |
| | Rice bran oil (excipient) | 500 |
| | Beeswax (excipient) | 30.36 |
| Shell | Fish gelatine | 267.7 |
| | Beta carotene colour | 8.6 |
| | Caramel colour (E150a) | 8.6 |

TABLE 10
Active ingredient of Triticum aestivum (wheat) grain extract in a single
softgel capsule containing the first composition, and its amount

| Ingredient | Weight (mg) |
|---|---|
| Ceramides | 3-6 mg |

TABLE 11
Ingredients and their amounts in a single softgel
capsule containing the second composition

| | Ingredient | Weight | |
|---|---|---|---|
| Filling | Fish oil | 570 | mg |
| | d alpha tocopherol (E306) | 1 | mg |
| | Oenothera biennis oil | 297 | mg |
| | Retinyl palmitate | 60 μg RAE* | |
| Shell | Fish gelatin | 267.99 | mg |
| | Glycerol (E422) | 107.598 | mg |

*μg RAE is μg retinol activity equivalent as defined herein.

TABLE 12
Active ingredients of fish oil in a single softgel capsule
containing the second composition, and their amounts

| Ingredient | Weight (mg) |
|---|---|
| Eicosapentaenoic acid (EPA) | 178 |
| Docosahexaenoic acid (DHA) | 119 |

TABLE 13
Active ingredient of Oenothera biennis oil in a single softgel
capsule containing the second composition, and its amount

| Ingredient | Weight (mg) |
|---|---|
| Gamma linoleic acid (GLA) | 25 |

### Results

Table 14 shows the results of Example 5 and compares them with those of Examples 3 and 4.

TABLE 14
Comparison between Examples 3-5

| | | Example 3 | Example 4 | Example 5 |
|---|---|---|---|---|
| | | Mean % | Mean % | Mean % |
| Parameter | Area | change | change | change |
| Hydration | Cheek | 64% | 40% | 82% |
| | | improvement | improvement | improvement |
| Skin | Cheek | 29% | Not measured | 11% |
| Roughness | | deterioration | | improvement |
| Wrinkle | Cheek | 18% | 12% | 17% |
| Depth | | deterioration | improvement | improvement |
| Wrinkle | Cheek | 16% | 5% | 19% |
| Width | | deterioration | improvement | improvement |
| Erythema | Cheek | Not measured | Not measured | 7% |
| | | | | improvement |

Skin roughness is a proxy for the rate at which skin cells die. Cell death leads to a thicker layer of dead cells at the skin surface which makes skin feel rougher. In Example 5, skin roughness improved by 11% on the cheek area while in Example 3, skin roughness worsened by 29%. It can be concluded that skin cell death may be mitigated by the composition of Example 5, compared to that of Example 3.

The improvement in wrinkle depth and width in Example 5 was surprising compared to Examples 3 and 4, suggesting a synergistic effect. A merely additive effect would have been a 6% deterioration in wrinkle depth and an 11% deterioration in wrinkle width, whereas what was actually observed in Example 5 was a 17% improvement in wrinkle depth and a 19% improvement in wrinkle width. It is notable that this difference is seen in the skin of the cheek; the cheek typically being the largest area of skin exposed to the environment and weather year-round. Without wishing to be bound by theory, these factors are thought to make the cheek an especially difficult area of skin for which to achieve improved hydration and reduced wrinkle depth and width. The cheek therefore provides the best ground for comparison between the results of Examples 3, 4 and 5. It is reasonable to conclude that there exists a surprising, synergistic effect on wrinkles, between the first and second composition (Example 5) compared to the compositions taken alone (Examples 3 and 4).

It was also surprising that wrinkle depth and width were reduced without skin irritation. Instead, skin became more hydrated and erythema decreased (7% improvement in erythema corresponding to a reduction in erythema).

The improvements of Example 5 were observed in a relatively short period of time, of only 12 weeks, i.e. about 3 months.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

What is claimed is:

1. A kit, comprising:
a first composition for oral administration, comprising hyaluronic acid, one or more ceramides and manganese; and
a second composition for oral administration, comprising retinol or a derivative thereof, one or more omega-3 fatty acids and one or more omega-6 fatty acids;
wherein the first composition comprises at least 20 mg hyaluronic acid; at least 1 mg of the one or more ceramides; and at least 0.1 mg manganese; and
wherein the second composition comprises at least 60 μg of retinol or a derivative thereof; at least 300 mg of one or more omega-3 fatty acids; and at least 20 mg of one or more omega-6 fatty acids.

2. The kit of claim 1, wherein the first composition comprises a compound selected from the group consisting of chondroitin sulphate, dermatan sulphate, derivatives thereof, and combinations thereof.

3. The kit of claim 2, wherein the first composition comprises at least 3 mg of said compound.

4. The kit of claim 1, wherein the first composition is provided in the form of at least one capsule; and wherein the second composition is provided in the form of at least one capsule.

5. A composition for oral administration, comprising hyaluronic acid, one or more ceramides, manganese, retinol or a derivative thereof, one or more omega-3 fatty acids, and one or more omega-6 fatty acids;
wherein the composition comprises at least 20 mg hyaluronic acid; at least 1 mg of the one or more ceramides; at least 0.1 mg manganese; at least 60 μg of retinol or a derivative thereof; at least 300 mg of one or more omega-3 fatty acids; and at least 20 mg of one or more omega-6 fatty acids.

6. The composition of claim 5, further comprising a compound selected from the group consisting of chondroitin sulphate, dermatan sulphate, derivatives thereof, and combinations thereof.

7. The composition of claim 6, comprising at least 3 mg of said compound.

8. A method of increasing skin hydration; and/or
reducing the depth and width of skin wrinkles, comprising administering to a human subject:
(a) a first composition, comprising hyaluronic acid, one or more ceramides and manganese; and
a second composition, comprising retinol or a derivative thereof, one or more omega-3 fatty acids and one or more omega-6 fatty acids; wherein the first composition comprises at least 20 mg hyaluronic acid; at least 1 mg of the one or more ceramides; and at least 0.1 mg manganese; and wherein the second composition comprises at least 60 μg of retinol or a derivative thereof; at least 300 mg of one or more omega-3 fatty acids; and at least 20 mg of one or more omega-6 fatty acids; or
(b) a third composition for oral administration, comprising hyaluronic acid, one or more ceramides, manganese, retinol or a derivative thereof, one or more omega-3 fatty acids, and one or more omega-6 fatty acids; wherein the third composition comprises at least 20 mg hyaluronic acid; at least 1 mg of the one or more ceramides; at least 0.1 mg manganese; at least 60 μg of retinol or a derivative thereof; at least 300 mg of one or more omega-3 fatty acids; and at least 20 mg of one or more omega-6 fatty acids.

9. The method of claim 8, further comprising:
orally administering to a human subject at least the first composition, comprising hyaluronic acid, one or more ceramides and manganese; and
orally administering to the human subject at least the second composition, comprising one or more omega-3 fatty acids, one or more omega-6 fatty acids and retinol;
wherein steps (i) and (ii) are each repeated at least once per day.

10. The method of claim 9, wherein the first composition is administered with food.

11. The method of claim 9, wherein the second composition is administered with food.

12. The method of claim 9, wherein said first composition and said second composition are administered with food.
